(19) United States
(12) Patent Application Publication
(10) Pub. No.: US 2010/0016830 A1
(43) Pub. Date: Jan. 21, 2010

(54) Injection of Anticoagulant Into Bone Marrow Space

(76) Inventor: James R. Ellsworth, Marshfield, MA (US)

Correspondence Address: CLARK & BRODY, 1090 VERMONT AVENUE, NW, SUITE 250, WASHINGTON, DC, 20005, US

(21) Appl. No.: 12/226,735

(22) PCT Filed: Apr. 16, 2007
(86) PCT No.: PCT/US2007/009356

§ 371 Date: May 22, 2009

Related U.S. Application Data
(60) Provisional application No. 60/795,553, filed on Apr. 28, 2006.

(51) Int. Cl. A61M 5/31; A61M 1/00

(52) U.S. Cl. 604/506

(57) ABSTRACT

A method for aspirating bone marrow includes the step of injecting anticoagulant into the area Of the bone marrow cavity surrounding the entry site of the aspiration needle. The amount of anticoagulant may be relatively small because the primary objective is to address the damage caused by entry of the needle into and through regions of the bone marrow cavity. As little as 0.5 ml heparin is injected prior to aspiration. Additional anticoagulant is provided in the collection syringe to facilitate further processing, as by centrifugal fractionation of the marrow to obtain desired components.

### TECHNICAL FIELD

This invention relates to the art of obtaining bone marrow, preferably, by aspiration techniques. In particular the invention relates to the use of anticoagulants during aspiration of bone marrow.

### BACKGROUND

It is known to collect peripheral blood and bone marrow aspirate for processing, for example by centrifugation, to fractionate the fluids into several cellular components. Peripheral blood is typically collected for this purpose with a minimum of anticoagulant, because it presents comparatively few clotting issues. Bone marrow aspirate, however, presents more serious clotting issues.

One reason for the presentation of more serious issues in the collection of bone marrow aspirate is that a simple and less-traumatic entry can be made to a blood vessel compared to the trauma caused by entering a bone marrow cavity and the marrow structure. Entry into a bone marrow cavity typically requires drilling a hole in the bone to receive a marrow aspiration needle, and this causes significant damage to the bone, the bone marrow, and the blood cells in the marrow cavity, all of which activate the clotting factors.

Typical bone marrow aspiration is performed as a biopsy, which requires only one or two cubic centimeters of marrow. These samples are, however, not appropriate for point-of-care fractionation because the volume retrieved is far too small. With increasing requests for clinical application of cells from marrow requiring point-of-care fractionation, new problems are apparent.

When collecting bone-marrow aspirate, the aspiration needle necessarily damages the marrow structure. Thus, it is not possible with known procedures to aspirate marrow that has not been damaged either by direct contact with the aspiration needle structure or by contact with damaged tissue. The clotting cascade is, thus, active in known bone marrow aspirate, which has resulted in the overuse of anti-coagulants in the marrow aspirate to prevent coagulation. Typical known anticoagulants are heparin, which interferes with the conversion of fibrinogen to fibrin, and citrate types such as ACD and CPD, which bind the calcium that is required throughout the clotting cascade.

While the present use of significant volumes of anticoagulant may be acceptable for laboratory processing, which washes and cultures the bone marrow, it is not acceptable for point-of-care treatment of a patient, because the high levels of anticoagulant have an undesirable effect on the harvested cells.

Moreover, in the typical aspiration of bone marrow, the anticoagulant is provided by placing it in the collection syringe. In this procedure, the first marrow that is aspirated into the syringe flows into the anticoagulant and interacts directly with the highly concentrated anticoagulant. This is undesirable because anticoagulant at high concentrations is highly detrimental to bone marrow cells. The marrow that enters the syringe subsequently, however, does not mix directly with the highly concentrated anticoagulant, the clotting cascade is further along due to the elapse of time, and the concentration of the anticoagulant is lower due to dilution by the additional marrow. Thus, the subsequent marrow is typically not anti-coagulated sufficiently. Further, as time elapses and clotting progresses, cells are activated and cell viability reduced.

Thus, there is a need for improved techniques in the aspiration of bone marrow.

### SUMMARY OF THE INVENTION

In accordance with the invention, the aspiration of bone marrow is greatly improved by injecting anticoagulant into the marrow cavity or space before the marrow is aspirated. It has been discovered that injection of only a small volume of anticoagulant into this space produces several advantages, as will be discussed below. It is believed that this results from the fact that the entry of the aspiration needle through the bone and into the marrow cavity causes significant damage to the area immediately surrounding the entry site but that treatment of this site with anticoagulant significantly reduces the effects of the damage. Thus, injection of even a small amount of anticoagulant into the area of the entry site retards the onset of the clotting cascade and allows aspiration of marrow having fewer damages cells.

Testing has shown that for centrifugation of bone marrow obtained from a horse in the typical prior art fashion, heparin levels as high as 80,000 u/60 ml was not sufficient to prevent clotting of the marrow aspirate during centrifugation. This high concentration of heparin, however, also causes the cells to be so fragile that they cannot be handled without significant lysing of the nucleated cells.

When using the technique according to the invention, however, clot-free marrow cells can be aspirated and processed successfully. For example, injecting 0.5 ml of 1000 u/ml heparin into the marrow space and using 2,000 u heparin/αml marrow provides clot free samples that can be processed and cultured successfully.

The volume of anticoagulant injected into the bone marrow cavity preferably approximates the volume of the damaged area in the cavity, and the concentration of the anticoagulant is determined to be that which reduces the effects of the damage to the site. For example, 0.4 ml to 1.2 ml anticoagulant may be injected in those instances where the aspiration needle is 8 gauge to 18 gauge, e.g., 11 gauge, and inserted about 2 cm into the cavity. Preferably, about 0.5 ml to 1.0 ml is injected and most preferably 0.5 ml. The figures given reflect the amount of anticoagulant actually injected and excludes the fluid that remains in the needle as “hold up.” It will be understood that the upper limits set forth may be exceeded if dilution of the aspirate is not of concern. Thus, many objectives of the invention can be achieved by injecting more anticoagulant than is described with the understanding that this will dilute the aspirate proportionately.

When heparin is the anticoagulant to be injected the concentration is preferably 500 u to 1000 u per milliliter. Heparin with a concentration of 5 u/ml, which is presently used for anticoagulation of peripheral blood, may, however, be used for bone marrow aspiration in accordance with the technique of the invention.

ACD may also be used as the anticoagulant. When used with prior techniques, this required more than forty percent ACD by volume to prevent clotting, which reduced the amount of marrow that could be processed. Injection of the anticoagulant into the marrow cavity in accordance with the invention; however, reduces the volume of anticoagulant required and increases the marrow processed.

Injecting anticoagulant into the bone marrow space before aspiration provides the advantage of increasing the flow rate of the aspirated marrow, thus reducing the time required for the aspiration procedure. Further, this technique reduces the number of marrow cells that come into contact with the clotting factors, which greatly improves the cell viability and allows collection of a greater number of cells (including platelets) with less activation. Moreover, cells aspirated in accordance with the technique of the invention do not require washing. Still further, the technique according to the invention facilitates point-of-care fractionation of the marrow because clotting is reduced, which eliminates such steps as filtration.

Point-of-care treatment of a patient with one or more components of bone marrow from that patient (i.e., autologous treatment) may be achieved by combining the method of the invention with the use of available systems such as the SmartPrep system offered by Harvest Technologies Corporation of Plymouth, Massachusetts.

An object of the invention is to provide efficient point-of-care treatment with bone marrow cells.

Another object of the invention is to improve the physiology of aspirated marrow cells.

Still another object of the invention is to reduce dilution of aspirated bone marrow cells.

### DETAILED DESCRIPTION OF THE PREFERRED EMBODIMENT

In accordance with the method of the present invention, a known aspiration needle is used to obtain access to a bone marrow cavity, and anticoagulant is injected into the bone marrow space. Subsequently, the marrow is aspirated in accordance with known techniques. The aspiration needle may be any of the known such instruments.

In the preferred embodiment, the anticoagulant injected into the cavity is heparin having a concentration of 1000 u/ml, but other known anticoagulants are contemplated, including but not limited to ACD as discussed above. Preferably, 0.5 ml of heparin at this concentration is injected into the area surrounding the entry site.

In addition, anticoagulant, such as heparin, is provided in a collection syringe into which the marrow is aspirated. Preferably, 2,000 u or less of heparin is provided in the collection syringe for about 60 ml marrow.

Modifications of the above method may be made without departing from the invention. For example, entry may be made into more than one site, and anticoagulant would be injected into each such site. As well, the aspiration needle may be moved from the initial site, which might require the injection of anticoagulant into the region surrounding the new location of the aspiration needle. In general, the invention contemplates injection of anticoagulant into the area surrounding the entry site where damage to the bone marrow cells might activate the cells and initiate the clotting cascade, but it is within the broader aspects of the invention to inject larger amounts of anticoagulant, for example, in those instances where the damage is expected to be greater or where the aspiration needle is expected to be moved in the marrow and sequential injections are not desired.

I claim:

1. The method of injecting anticoagulant into a bone marrow cavity and aspirating marrow to reduce the amount of anticoagulant required.

2. A method of aspirating bone marrow comprising obtaining access to a bone marrow cavity, injecting anticoagulant into said cavity, and aspirating bone marrow from said cavity.

3. A method according to claim 2 wherein said anticoagulant is heparin.

4. A method according to claim 2 wherein said anticoagulant is ACD.

5. A method according to claim 2 wherein said anticoagulant is CPD.

6. A method according to claim 2 wherein said step of injecting anticoagulant includes the step of injecting about 0.4 ml to about 1.2 ml heparin.

7. A method according to claim 6 further comprising the step of providing additional anticoagulant in a collection chamber.

8. A method according to claim 7 wherein said additional anticoagulant is less than about 2000 u heparin per 60 ml of aspirated bone marrow.

9. A method according to claim 7 further comprising the step of centrifugally separating components of the aspirated bone marrow.

10. A method according to claim 6 wherein said step of injecting comprises injecting about 0.5 ml to about 1.0 ml heparin.

11. A method according to claim 10 wherein said step of injecting comprises injecting at least about 0.4 ml heparin having a concentration of at least about 500 u/ml.

12. A method according to claim 10 wherein said step of injecting comprises injecting at least about 0.5 ml heparin having a concentration of about 1000 u/ml.

13. A method according to claim 2 further comprising separating said bone marrow that has been aspirated into components by centrifugation and treating a patient with at least one of said components.
